(19) United States
(12) Patent Application Publication
(10) Pub. No.: US 2012/0107894 A1
(43) Pub. Date: May 3, 2012

(54) Methods and Compositions for the Recombinant Biosynthesis of Terminal Olefins

(75) Inventors: Frank A. Skraly, Watertown, MA (US); Christian P. Ridley, Acton, MA (US)

(73) Assignee: JOULE UNLIMITED TECHNOLOGIES, INC., Cambridge, MA (US)

(21) Appl. No.: 13/284,311

(22) Filed: Oct. 28, 2011

Related U.S. Application Data
(60) Provisional application No. 61/407,699, filed on Oct. 28, 2010.

(51) Int. Cl. C12P 5/00; C12N 1/19; C12N 1/12; C12N 1/21

(52) U.S. Cl. 435/166; 435/252.3; 435/252.33; 435/254.2; 435/254.21; 435/257.2

(57) ABSTRACT

The present disclosure identifies methods and compositions for modifying microbial cells, such that the organisms efficiently synthesize terminal olefins, and in particular the use of such organisms for the commercial production of propylene and related molecules.

### SEQUENCE LISTING

The instant application contains a Sequence Listing which has been submitted in ASCII format via EFS-Web and is hereby incorporated by reference in its entirety. Said ASCII copy, created on Oct. 28, 2011, is named 19357US_CRF_sequencelisting.txt and is 909,261 bytes in size.

### FIELD OF THE INVENTION

The present disclosure relates to methods for conferring terminal olefin-producing properties to a heterotrophic or photoautotrophic microbial cell, such that the modified microbial cells can be used in the commercial production of terminal olefins.

### BACKGROUND OF THE INVENTION

A terminal olefin is an unsaturated organic compound with a carbon chain backbone, having at least one double bond at the end of the carbon chain. Synthesis of terminal olefins, such as propylene, has significant utility from an industrial prospective.

Propylene is a terminal olefin molecule of chemical formula C₃H₆ which is used to manufacture polyethylene, polypropylene, alpha olefins, and styrene. It is also used industrially to produce materials such as polyester, acrylics, ethylene glycol antifreeze, polyvinyl chloride (PVC), propylene oxide, oxo alcohols, and isopropanol. Propylene can be derived from fractional distillation from hydrocarbon mixtures obtained from cracking and other refining processes. However, propylene production by engineered host cells represents a significant alternative to traditional methods of production.

A need exists therefore, for photosynthetic and non-photosynthetic strains which can make terminal olefins such as propylene and related molecules.

### SUMMARY OF THE INVENTION

The disclosure provides a microbial cell for producing a hydrocarbon comprising a recombinant sulfotransferase protein activity and/or a recombinant thioesterase protein activity, wherein the cell synthesizes at least one terminal olefin. The disclosure further provides a method for producing a terminal olefin, comprising culturing an engineered microbial cell in a culture medium, wherein the engineered microbial cell comprises a set of recombinant enzymes comprising at least one sulfotransferase domain and/or at least one thioesterase domain; and isolating the terminal olefin from the microbial cell or the culture medium. In one embodiment of the invention, the microbial cell comprises a nonA gene. In another embodiment, the microbial cell comprises a recombinantly expressed protein comprising any of SEQ ID NOs: 1-3. In an alternative embodiment, the microbial cell comprises a recombinantly expressed protein selected from Tables 1-3 (SEQ ID NOS 4-104, respectively, in order of appearance).

In one aspect of the invention, the microbial cell is a gram-negative or gram-positive bacterium. In another aspect of the invention, the microbial cell is capable of photosynthesis. In still another aspect, the microbial cell is a cyanobacterium. In yet another aspect, the microbial cell comprises endogenous 3-hydroxybutyryl-ACP and/or endogenous 3-hydroxybutyryl-CoA.

In one embodiment, the microbial cell is engineered to synthesize 3-hydroxybutyryl-ACP. In another embodiment, the engineering comprises expressing in the microbial cell a recombinant accBCAD gene or a recombinant fabDHG gene. In still another embodiment, the engineering comprises expressing in said microbial cell a genetically modified gene encoding a polypeptide comprising 3-hydroxyacyl-ACP dehydratase activity. In a further embodiment, the engineered microbial cell has a reduced 3-hydroxyacyl-ACP dehydratase activity as compared to a control microbial cell that does not express the genetically modified gene encoding a polypeptide comprising 3-hydroxyacyl-ACP dehydratase activity. In still another embodiment, the genetic modification knocks out an endogenous gene encoding a polypeptide comprising 3-hydroxyacyl-ACP dehydratase activity. In yet another embodiment, the genetically modified gene encoding a polypeptide comprising 3-hydroxyacyl-ACP dehydratase activity is under the control of an inducible promoter. In another embodiment, the microbial cell is cultured in the presence of long chain fatty acids. In one embodiment, the microbial cell produces propylene.

The invention provides for a microbial cell engineered to synthesize 3-hydroxybutyryl-CoA. The invention also provides for a microbial cell engineered to express recombinant phaA gene and a recombinant phaB gene. In one embodiment, the microbial cell produces propylene. In another embodiment, the propylene is synthesized from acetyl-CoA. In still another embodiment, the terminal olefin synthesized in the microbial cell is selected from the group consisting of ethylene, propylene, butylenes, butadiene, isoprene, and 1-nonadecene.

In one particular embodiment, the microbial cell recombinantly expresses a curM gene. In another particular embodiment, the microbial cell recombinantly expresses a nonA gene.

In another embodiment of the present invention, an engineered microbial cell is provided, wherein the engineered microbial cell is selected from the group consisting of a bacterium, a yeast, and an algae, wherein the engineered microbial cell comprises one or more recombinant genes encoding a polypeptide comprising a sulfotransferase domain and/or a thioesterase domain, and wherein the engineered microbial cell synthesizes at least one terminal olefin. In a further embodiment, the bacterium is cyanobacterium. In another further embodiment, the bacterium is E. Coli. In yet another embodiment, the bacterium is Chlamydomonas reinhardtii. In still another embodiment, the bacterium is Chlamydomonas reinhardtii. In one particular embodiment, the yeast is S. cerevisiae.

### BRIEF DESCRIPTION OF THE FIGURES

FIG. 1: Pathway for synthesis of propylene from 3-hydryxobutyryl-CoA or 3-hydroxybutyryl-ACP.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same microbial cell or progeny thereof) or exogenous (originating from a different microbial cell or progeny thereof). By way of example, a promoter sequence can be substituted (e.g., by homologous recombination) for the native promoter of a gene in the genome of a microbial cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a microbial cell chromosome at a heterologous site and a nucleic acid construct present as an episome.

The nucleic acids (also referred to as polynucleotides) of the present invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

Deletion: The removal of one or more nucleotides from a nucleic acid molecule or one or more amino acids from a protein, the regions on either side being joined together.

Knock-out: A gene whose level of functional expression or activity has been reduced to an undetectable levels. In some examples, a gene is knocked-out via deletion of some or all of its coding sequence. In other examples, a gene is knocked-out via introduction of one or more nucleotides into its open-reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant microbial cell” (or simply “microbial cell” or “host cell”), as used herein, is intended to refer to a cell into which a recombinant nucleic acid molecule, such as, e.g., a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “microbial cell” or “host cell” as used herein. A recombinant microbial cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal, an internal, and/or a carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein may comprise at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of any of the proteins of the present invention have particular utility. The heterologous polypeptide included within the fusion protein of an embodiment of the present invention is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present invention may be used to produce an equivalent effect and are therefore envisioned to be part of an embodiment of the present invention.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, a cytoplasmic domain, a thioesterase domain, and a sulfotransferase domain.

The term thioesterase activity or “TE” refers to an enzymatic activity of a polypeptide which catalyzes the hydrolytic cleavage of energy-rich thioester bonds as in acetyl-CoA. This activity is useful in the catalytic conversion of 3-hydroxybutyryl-CoA or 3-hydroxybutyryl-ACP to propylene.

The term sulfotransferase activity or “ST” refers to an enzymatic activity of a polypeptide which catalyzes the transfer of a sulfate group from one compound to the hydroxyl group of another. This activity is useful in the catalytic conversion of 3-hydroxybutyryl-CoA or 3-hydroxybutyryl-ACP to propylene.

As used herein, the term “molecule” means any compound, including, but not limited to, a small molecule, peptide, protein, sugar, nucleotide, nucleic acid, lipid, etc., and such a compound can be natural or synthetic.

Biofuel: A biofuel is any fuel that derives from a biological source. Biofuel refers to one or more hydrocarbons, one or more alcohols, one or more fatty esters or a mixture thereof. Preferably, liquid hydrocarbons are used.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Terminal Olefin: a terminal olefin is an olefin (or alkene) having at least one carbon-carbon double bond located at the terminal end of the carbon chain backbone. Terminal olefins are unsaturated hydrocarbons. They can be straight chain, branched, and cyclic terminal olefins.

Propylene or Propene: is an unsaturated organic compound having the chemical formula C₃H₆. It has one double bond, and is the second simplest member of the alkene class of hydrocarbons.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Throughout this specification and claims, the word “comprise” or variations such as “comprises” or “comprising”, in association with a numeric limitation, including a numeric range, will be understood to imply the inclusion of a stated integer or group of integers but not the exclusion of any other integer or group of integers.

### Nucleic Acid Sequences

Terminal olefins are chemical compounds that consist only of the elements carbon (C) and hydrogen (H) (i.e., hydrocarbons), containing at least carbon-carbon double bond (i.e., they are unsaturated compounds). Together, thioesterase (TE) and sulfotransferase (ST) enzymes function to synthesize terminal olefins, such as propylene from acetyl-CoA molecules and other precursors.

Accordingly, an embodiment of the present invention provides isolated nucleic acid molecules for genes encoding TE and ST enzymes, and variants thereof. In one embodiment, the present invention provides an isolated nucleic acid molecule having a nucleic acid sequence comprising or consisting of a gene coding for TE and ST, and homologs, variants and derivatives thereof expressed in a host cell of interest. An embodiment of the present invention also provides a nucleic acid molecule comprising or consisting of a sequence which is a codon and expression optimized version of the TE and ST genes described herein. In a further embodiment, the present invention provides a nucleic acid molecule and homologs, variants and derivatives of the molecule comprising or consisting of a sequence which is a variant of the TE and ST gene having at least 76% sequence identity to a wild-type gene. The nucleic acid sequence can be preferably 80%, 85%, 90%, 95%, 98%, 99%, 99.9% or even higher identity to the wild-type gene. In one embodiment, the nucleic acid sequence encodes an enzyme selected from Tables 1-3 (SEQ ID NOS 4-104, respectively, in order of appearance).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

Another embodiment of the invention also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

As is well known in the art, enzyme activities can be measured in various ways. For example, the activity of the enzyme can be followed using chromatographic techniques, such as by high performance liquid chromatography. Chung and Sloan, J. Chromatogr. 371:71-81 (1986). As another alternative the activity can be indirectly measured by determining the levels of product made from the enzyme activity. These levels can be measured with techniques including aqueous chloroform/methanol extraction as known and described in the art (Cf. M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y.: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G., R. O. Dunn, and M. O. Bagby. 1997. Biodiesel: The use of vegetable oils and their derivatives as alternative diesel fuels. Am. Chem. Soc. Symp. Series 666: 172-208), titration for determining free fatty acids (Komers, K., F. Skopal, and R. Stloukal. 1997. Determination of the neutralization number for biodiesel fuel production. Fett/Lipid 99(2): 52-54), enzymatic methods (Bailer, J., and K. de Hueber. 1991. Determination of saponifiable glycerol in “bio-diesel.” Fresenius J. Anal. Chem. 340(3): 186), physical property-based methods, wet chemical methods, etc. can be used to analyze the levels and the identity of the product produced by the organisms of an embodiment of the present invention. Other methods and techniques may also be suitable for the measurement of enzyme activity, as would be known by one of skill in the art.

### Plasmids

Plasmids relevant to genetic engineering typically include at least two functional elements 1) an origin of replication enabling propagation of the DNA sequence in the host organism, and 2) a selective marker (for example an antibiotic resistance marker conferring resistance to ampicillin, kanamycin, zeocin, chloramphenicol, tetracycline, spectinomycin, and the like). Plasmids are often referred to as “cloning vectors” when their primary purpose is to enable propagation of a desired heterologous DNA insert. Plasmids can also include cis-acting regulatory sequences to direct transcription and translation of heterologous DNA inserts (for example, promoters, transcription terminators, ribosome binding sites); such plasmids are frequently referred to as “expression vectors.” When plasmids contain functional elements that allow for propagation in more than one species, such plasmids are referred to as “shuttle vectors.” Shuttle vectors are well known to those in the art. For example, pSE4 is a shuttle vector that allows propagation in E. coli and Synechococcus [Maeda S, Kawaguchi Y, Ohy T, and Omata T. J. Bacteriol. (1998). 180:4080-4088]. Shuttle vectors are particularly useful in one embodiment of the present invention to allow for facile manipulation of genes and regulatory sequences.

### Vectors

Also provided are vectors, including expression vectors and cloning vectors, which comprise the above nucleic acid molecules of an embodiment of the present invention. In a first embodiment, the vectors include the isolated nucleic acid molecules described above. In an alternative embodiment, the vectors include the above-described nucleic acid molecules operably linked to one or more expression control sequences. The vectors of the instant invention may thus be used to express an ST and/or TE polypeptide contributing to polypropylene producing activity by a host cell.

Exemplary vectors of the invention include any of the vectors expressing a thioesterase or sulfotranserase. A gene expressing a thioesterase or sulfotransferase are assembled and inserted into a suitable vector, e.g. pJB5, as described in WO2009/111513, herein incorporated in its entirety by reference. The invention also provides other vectors such as pJB161, as described in WO2009/062190 and U.S. Pat. No. 7,785,861, herein incorporated in their entirety by reference, which are capable of receiving nucleic acid sequences of the invention. Vectors such as pJB161 comprise sequences which are homologous with sequences that are present in plasmids which are endogenous to certain photosynthetic microorganisms (e.g., plasmids pAQ7 or pAQ1 of certain Synechococcus species). Recombination between pJB161 and the endogenous plasmids in vivo yield engineered microbes expressing the genes of interest from their endogenous plasmids. Alternatively, vectors can be engineered to recombine with the host cell chromosome, or the vector can be engineered to replicate and express genes of interest independent of the host cell chromosome or any of the host cell's endogenous plasmids.

Vectors useful for expression of nucleic acids in prokaryotes are well known in the art.

### Isolated Polypeptides

According to another aspect of the present invention, isolated polypeptides (including muteins, allelic variants, fragments, derivatives, and analogs) encoded by the nucleic acid molecules are provided. In one embodiment, isolated polypeptides comprising a fragment of the above-described polypeptide sequences are provided. These fragments preferably include at least 20 contiguous amino acids, more preferably at least 25, 30, 35, 40, 45, 50, 60, 70, 80, 90, 100 or even more contiguous amino acids.

The polypeptides of an embodiment of the present invention also include fusions between the above-described polypeptide sequences and heterologous polypeptides. The heterologous sequences can, for example, include sequences designed to facilitate purification, e.g. histidine tags, and/or visualization of recombinantly-expressed proteins. Other non-limiting examples of protein fusions include those that permit display of the encoded protein on the surface of a phage or a cell, fusions to intrinsically fluorescent proteins, such as green fluorescent protein (GFP), and fusions to the IgG Fc region.

### Host Cell Transformants

In another aspect of the present invention, host cells transformed with the nucleic acid molecules or vectors of an embodiment of the present invention, and descendants thereof, are provided. In some embodiments of the present invention, these cells carry the nucleic acid sequences of an embodiment of the present invention on vectors, which may but need not be freely replicating vectors. In other embodiments of the present invention, the nucleic acids have been integrated into the genome of the host cells.

In a preferred embodiment, the host cell comprises one or more ST and/or TE encoding nucleic acids which express ST and/or TE activity in the host cell.

In an alternative embodiment, the host cells of an embodiment of the present invention are mutated by recombination with a disruption, deletion or mutation of the isolated nucleic acid of the present invention so that the activity of the ST and/or TE protein(s) in the host cell is reduced or eliminated compared to a host cell lacking the mutation.

### Selected or Engineered Microorganisms For the Production of Carbon-Based Products of Interest

Microorganism: Includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure tolerant organisms include piezophiles, which tolerate pressure of 130 MPa. Weight tolerant organisms include barophiles. Hypergravity (e.g., >1 g) hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt tolerant organisms include halophiles (e.g., 2-5 M NaCl) Halobacteriacea and Dunaliella salina. pH tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New York: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera:

Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera:

Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis.

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S°-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

HyperPhotosynthetic conversion requires extensive genetic modification; thus, in preferred embodiments the parental photoautotrophic organism can be transformed with exogenous DNA.

Preferred organisms for HyperPhotosynthetic conversion include: Arabidopsis thaliana, Panicum virgatum, Miscanthus giganteus, and Zea mays (plants), Botryococcus braunii, Chlamydomonas reinhardtii and Dunaliela salina (algae), Synechococcus sp PCC 7002, Synechococcus sp. PCC 7942, Synechocystis sp. PCC 6803, and Thermosynechococcus elongatus BP-1 (cyanobacteria), Chlorobium tepidum (green sulfur bacteria), Chloroflexus auranticus (green non-sulfur bacteria), Chromatium tepidum and Chromatium vinosum (purple sulfur bacteria), Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A common theme in selecting or engineering a suitable organism is autotrophic fixation of CO₂ to products. This would cover photosynthesis and methanogenesis. Acetogenesis, encompassing the three types of CO₂ fixation; Calvin cycle, acetyl CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

The host cell of one embodiment of the present invention is preferably Escherichia coli, Synechococcus, Thermosynechococcus, Synechocystis, Klebsiella oxytoca, or Saccharomyces cerevisiae but other prokaryotic, archaea and eukaryotic host cells including those of the cyanobacteria are also encompassed within the scope of the present invention.

### Hydroxyacyl Substrates

The compositions and methods described herein can be used to produce olefins (e.g., terminal olefins) from hydroxyacyl substrates. While not wishing to be bound by theory it is believed that the polypeptides described herein produce olefins from hydroxyacyl substrates via a sulfotransferase and thioesterase mechanism. Thus, olefins having particular branching patterns, levels of saturation, and carbon chain length can be produced from hydroxyacyl substrates having those particular characteristics. Accordingly, each step within a hydroxyacyl related pathway can be modified to produce or overproduce a hydroxyacyl substrate of interest.

### Producing Terminal Olefins Using Cell-Free Methods

Some methods described herein, a terminal olefin can be produced using a purified polypeptide described herein and a hydroxyacyl substrate. For example, a host cell can be engineered to express a polypeptide (e.g. a NonA polypeptide or a variant thereof) as described herein. The host cell can be cultured under conditions suitable to allow expression of the polypeptide. Cell free extracts can then be generated using known methods. For example, the host cells can be lysed using detergents or by sonication. The expressed polypeptides can be purified using known methods. After obtaining the cell free extracts, hydroxyacyl substrates described herein can be added to the cell free extracts and maintained under conditions to allow conversion of hydroxyacyl substrates to terminal olefins. The terminal olefins can be separated and purified using known techniques.

The following examples are for illustrative purposes and are not intended to limit the scope of the present invention.

### Example 1

### A Pathway for the Enzymatic Synthesis of Terminal Olefins from 3-Hydroxyacyl Substrates

The nonA gene in Synechococcus elongatus PCC 7002 has been discovered by us to be responsible for synthesis of 1-nonadecene and other long-chain terminal olefins, as described in PCT/US2010/039558, herein incorporated by reference in its entirety. This newly discovered enzymatic activity is attributed to ST and TE domains present in the enzyme expressed by this gene. In this example, we express ST and TE domains of a protein such as L. majuscula CurM or S. elongatus PCC 7002 NonA in a host cell to convert 3-hydroxyacyl substrates to the corresponding terminal olefins, e.g. propylene.

### Example 2

### A Pathway for the Enzymatic Synthesis of Propylene

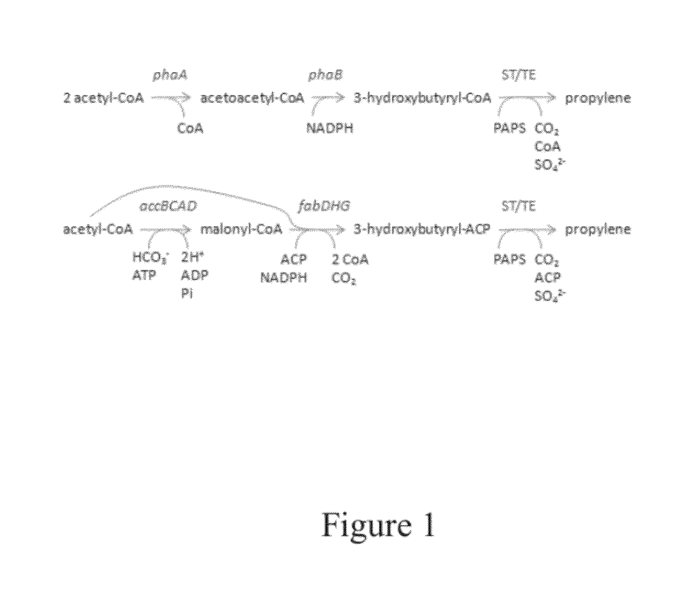

In this example, we use recombinant or endogenous ST and TE activity to convert 3-hydroxybutyryl-ACP or 3-hydroxybutyryl-CoA to propylene and CO₂ with the help of the cofactor 3′-phosphate 5′-phosphosulfate (PAPS), which occurs widely in bacterial and other biological systems (FIG. 1).

To obtain 3-hydroxybutyryl-CoA, we express R. eutropha phaA and phaB in the host cell, whose gene products together convert 2 acetyl-CoA molecules to 3-hydroxybutyryl-CoA and CoA, using NADPH as a cofactor.

To obtain 3-hydroxybutyryl-ACP, we utilize a host with attenuated 3-hydroxyacyl-ACP dehydratase (EC 4.2.1.59 and/or EC 4.2.1.58) activity while feeding long-chain fatty acids to enable lipid synthesis. In an alternative embodiment, the 3-hydroxyacyl-ACP dehydratase is placed under inducible control and expressed only under growth conditions. This allows fatty acid biosynthesis to proceed only to 3-hydroxybutyryl-ACP while still allowing the cell to grow. In this way, one obtains a pathway from acetyl-CoA to propylene.

### Example 3

### Homologous ST and TE Domains

The sequences of the ST and TE domains of the Synechococcus elongatus sp. PCC7002 NonA protein (SEQ ID NO:1) were used to perform an amino acid sequence search for homologous proteins using BLAST. Proteins homologous to the region of the protein comprising both ST and TE domains are listed in Table 1 (SEQ ID NOS 4-11, respectively, in order of appearance). Sequences homologous to only the NonA ST domain protein sequence (SEQ ID NO:2) are listed in Table 2 (SEQ ID NOS 12-19, respectively, in order of appearance). Sequences homologous to only the NonA TE domain protein sequence (SEQ ID NO:3) are listed in Table 3 (SEQ ID NOS 20-104, respectively, in order of appearance). At least one of the protein sequences of Tables 1-3 (SEQ ID NOS 4-104, respectively, in order of appearance) is engineered into a host cell, e.g. cyanobacterium, according to standard genetic engineering techniques. The engineered host cell has an increased capacity to synthesize terminal olefins, e.g. propylene.

TABLE 1
Proteins showing homology to both ST and TE domains of NonA.

| SEQ ID NO: | Protein ID | GenBank-annotated function | Organism |
|---|---|---|---|
| 4 | YP_001734428.1 | polyketide synthase | Synechococcus sp. PCC 7002 |
| 5 | YP_002377174.1 | beta-ketoacyl synthase | Cyanothece sp. PCC 7424 |
| 6 | YP_003887107.1 | beta-ketoacyl synthase | Cyanothece sp. PCC 7822 |
| 7 | ACV42478.1 | polyketide synthase | Lyngbya majuscula 19L |
| 8 | AAT70108.1 | CurM | Lyngbya majuscula |
| 9 | YP_610919.1 | polyketide synthase | Pseudomonas entomophila L48 |
| 10 | YP_003265308.1 | KR domain protein | Haliangium ochraceum DSM |
| | | | 14365 |
| 11 | XP_002507643.1 | modular polyketide synthase | Micromonas sp. RCC299 |
| | | type I | |

TABLE 2
Proteins showing homology to only the ST domain of NonA.

| SEQ ID NO: | Protein ID | GenBank-annotated function | Organism |
|---|---|---|---|
| 12 | YP_001062692.1 | CurM | Burkholderia pseudomallei 668 |
| 13 | ABW84363.1 | OciA | Planktothrix agardhii NIES-205 |
| 14 | ABI26077.1 | OciA | Planktothrix agardhii NIVA-CYA |
| | | | 116 |
| 15 | YP_003137597.1 | amino acid adenylation | Cyanothece sp. PCC 8802 |
| | | domain protein | |
| 16 | YP_002372038.1 | amino acid adenylation | Cyanothece sp. PCC 8801 |
| | | domain protein | |
| 17 | XP_003074830.1 | COG3321: Polyketide | Ostreococcus tauri |
| | | synthase modules and related | |
| | | proteins (ISS) | |
| 18 | XP_001416378.1 | polyketide synthase | Ostreococcus lucimarinus |
| | | | CCE9901 |
| 19 | ZP_03631565.1 | amino acid adenylation | bacterium Ellin514 |
| | | domain protein | |

TABLE 3
Proteins showing homology to only the TE domain of NonA.

| SEQ ID NO: | Protein ID | GenBank-annotated function | Organism |
|---|---|---|---|
| 20 | YP_001734428.1 | polyketide synthase | Synechococcus sp. PCC |
| | | | 7002 |
| 21 | AAC14106.1 | epoxide hydroxylase | Synechococcus sp. PCC |
| | | | 7002 |
| 22 | YP_433651.1 | alpha/beta superfamily | Hahella chejuensis KCTC |
| | | hydrolase/acyltransferase | 2396 |
| 23 | YP_001769292.1 | alpha/beta hydrolase fold | Methylobacterium sp. 4- |
| | | | 46 |
| 24 | YP_003269090.1 | alpha/beta hydrolase fold protein | Haliangium ochraceum |
| | | | DSM 14365 |
| 25 | ZP_01916760.1 | Alpha/beta hydrolase fold protein | Limnobacter sp. MED105 |
| 26 | YP_933620.1 | hydrolase or acytransferase | Azoarcus sp. BH72 |
| 27 | YP_158988.1 | putative hydrolase | Aromatoleum aromaticum |
| | | | EbN1 |
| 28 | YP_003776671.1 | hydrolase | Herbaspirillum |
| | | | seropedicae SmR1 |
| 29 | BAI49930.1 | putative esterase | uncultured microorganism |
| 30 | YP_662370.1 | alpha/beta hydrolase fold | Pseudoalteromonas |
| | | | atlantica T6c |
| 31 | ZP_01459983.1 | lipase A | Stigmatella aurantiaca |
| | | | DW4/3-1 |
| 32 | YP_634109.1 | alpha/beta fold family hydrolase | Myxococcus xanthus DK |
| | | | 1622 |
| 33 | ZP_01615147.1 | alpha/beta hydrolase | marine gamma |
| | | | proteobacterium |
| | | | HTCC2143 |
| 34 | YP_001352966.1 | alpha/beta fold family hydrolase | Janthinobacterium sp. |
| | | | Marseille |
| 35 | ZP_01307598.1 | hydrolase, alpha/beta fold family | Oceanobacter sp. RED65 |
| | | protein | |
| 36 | YP_001100441.1 | putative hydrolase protein | Herminiimonas |
| | | | arsenicoxydans |
| 37 | EFP65715.1 | alpha/beta hydrolase family protein | Ralstonia sp. 5_7_47FAA |
| 38 | YP_002981038.1 | alpha/beta hydrolase fold protein | Ralstonia pickettii 12D |
| 39 | YP_001898558.1 | alpha/beta hydrolase fold | Ralstonia pickettii 12J |
| 40 | YP_001172415.1 | hydrolase | Pseudomonas stutzeri |
| | | | A1501 |
| 41 | YP_002354112.1 | alpha/beta hydrolase fold protein | Thauera sp. MZ1T |
| 42 | ZP_05040720.1 | hydrolase, alpha/beta fold family, | Alcanivorax sp. DG881 |
| | | putative | |
| 43 | YP_001021961.1 | putative hydrolase protein | Methylibium |
| | | | petroleiphilum PM1 |
| 44 | YP_002030374.1 | alpha/beta hydrolase fold | Stenotrophomonas |
| | | | maltophilia R551-3 |
| 45 | ZP_01126880.1 | Alpha/beta hydrolase fold protein | Nitrococcus mobilis Nb- |
| | | | 231 |
| 46 | YP_001974273.1 | putative alpha/beta fold hydrolase | Stenotrophomonas |
| | | family protein | maltophilia K279a |
| 47 | YP_286430.1 | Alpha/beta hydrolase fold | Dechloromonas aromatica |
| | | | RCB |
| 48 | YP_001990203.1 | alpha/beta hydrolase fold | Rhodopseudomonas |
| | | | palustris TIE-1 |
| 49 | YP_917027.1 | alpha/beta hydrolase fold | Paracoccus denitrificans |
| | | | PD1222 |
| 50 | YP_002005206.1 | putative Alpha/beta fold hydrolase | Cupriavidus taiwanensis |
| 51 | YP_283592.1 | Alpha/beta hydrolase fold | Dechloromonas aromatica |
| | | | RCB |
| 52 | YP_001349005.1 | putative hydrolase | Pseudomonas aeruginosa |
| | | | PA7 |
| 53 | YP_001187947.1 | alpha/beta hydrolase fold | Pseudomonas mendocina |
| | | | ymp |
| 54 | ZP_04576152.1 | hydrolase | Oxalobacter formigenes |
| | | | HOxBLS |
| 55 | NP_250313.1 | probable hydrolase | Pseudomonas aeruginosa |
| | | | PAO1 |
| 56 | NP_900963.1 | hydrolase | Chromobacterium |
| | | | violaceum ATCC 12472 |
| 57 | AAT50924.1 | PA1622 | synthetic construct |
| 58 | YP_725707.1 | alpha/beta superfamily | Ralstonia eutropha H16 |
| | | hydrolase/acyltransferase | |
| 59 | YP_001554328.1 | alpha/beta hydrolase fold | Shewanella baltica OS195 |
| 60 | YP_002441288.1 | putative hydrolase | Pseudomonas aeruginosa |
| | | | LESB58 |
| 61 | YP_693203.1 | hydrolase | Alcanivorax borkumensis |
| | | | SK2 |
| 62 | YP_002798221.1 | alpha/beta hydrolase | Azotobacter vinelandii DJ |
| 63 | NP_001079604.1 | serine hydrolase-like 2 | Xenopus laevis |
| 64 | NP_946347.1 | Alpha/beta hydrolase fold | Rhodopseudomonas |
| | | | palustris CGA009 |
| 65 | YP_870022.1 | alpha/beta hydrolase fold | Shewanella sp. ANA-3 |
| 66 | YP_295320.1 | Alpha/beta hydrolase fold | Ralstonia eutropha |
| | | | JMP134 |
| 67 | YP_001982425.1 | hydrolase, alpha/beta fold family | Cellvibrio japonicus |
| | | | Ueda107 |
| 68 | YP_963643.1 | alpha/beta hydrolase fold | Shewanella sp. W3-18-1 |
| 69 | ZP_06358651.1 | alpha/beta hydrolase fold protein | Rhodopseudomonas |
| | | | palustris DX-1 |
| 70 | YP_001366096.1 | alpha/beta hydrolase fold | Shewanella baltica OS185 |
| 71 | ZP_01707636.1 | alpha/beta hydrolase fold | Shewanella putrefaciens |
| | | | 200 |
| 72 | YP_734308.1 | alpha/beta hydrolase fold | Shewanella sp. MR-4 |
| 73 | ZP_04957287.1 | hydrolase | gamma proteobacterium |
| | | | NOR51-B |
| 74 | NP_718168.1 | alpha/beta fold family hydrolase | Shewanella oneidensis |
| | | | MR-1 |
| 75 | YP_003146580.1 | alpha/beta hydrolase fold protein | Kangiella koreensis DSM |
| | | | 16069 |
| 76 | YP_568320.1 | alpha/beta hydrolase fold | Rhodopseudomonas |
| | | | palustris BisB5 |
| 77 | YP_001183284.1 | alpha/beta hydrolase fold | Shewanella putrefaciens |
| | | | CN-32 |
| 78 | ZP_05134273.1 | hydrolase of the alpha/beta fold | Stenotrophomonas sp. |
| | | superfamily | SKA14 |
| 79 | YP_003545632.1 | putative alpha/beta hydrolase | Sphingobium japonicum |
| | | | UT26S |
| 80 | YP_002358347.1 | alpha/beta hydrolase fold protein | Shewanella baltica OS223 |
| 81 | YP_856727.1 | alpha/beta fold family hydrolase | Aeromonas hydrophila |
| | | | subsp. hydrophila ATCC |
| | | | 7966 |
| 82 | XP_003055946.1 | predicted protein | Micromonas pusilla |
| | | | CCMP1545 |
| 83 | ZP_01616002.1 | putative hydrolase | marine gamma |
| | | | proteobacterium |
| | | | HTCC2143 |
| 84 | YP_001411669.1 | alpha/beta hydrolase fold | Parvibaculum |
| | | | lavamentivorans DS-1 |
| 85 | ZP_07392985.1 | alpha/beta hydrolase fold protein | Shewanella baltica OS183 |
| 86 | YP_001050238.1 | alpha/beta hydrolase fold | Shewanella baltica OS155 |
| 87 | YP_002553684.1 | alpha/beta hydrolase fold protein | Acidovorax ebreus TPSY |
| 88 | YP_003165824.1 | alpha/beta hydrolase fold protein | Candidatus |
| | | | Accumulibacter |
| | | | phosphatis clade IIA str. |
| | | | UW-1 |
| 89 | YP_001141910.1 | alpha/beta fold family hydrolase | Aeromonas salmonicida |
| | | | subsp. salmonicida A449 |
| 90 | ZP_04579173.1 | hydrolase | Oxalobacter formigenes |
| | | | OXCC13 |
| 91 | YP_001502304.1 | alpha/beta hydrolase fold | Shewanella pealeana |
| | | | ATCC 700345 |
| 92 | YP_484670.1 | Alpha/beta hydrolase | Rhodopseudomonas |
| | | | palustris HaA2 |
| 93 | YP_001615653.1 | putative hydrolase | Sorangium cellulosum ‘So |
| | | | ce 56’ |
| 94 | YP_003752880.1 | putative Alpha/beta fold hydrolase | Ralstonia solanacearum |
| | | | PSI07 |
| 95 | XP_002192434.1 | PREDICTED: serine hydrolase-like 2 | Taeniopygia guttata |
| 96 | YP_235108.1 | Alpha/beta hydrolase fold | Pseudomonas syringae pv. |
| | | | syringae B728a |
| 97 | YP_002795270.1 | Probable hydrolase | Laribacter hongkongensis |
| | | | HLHK9 |
| 98 | XP_001749708.1 | hypothetical protein | Monosiga brevicollis MX1 |
| 99 | YP_274221.1 | lipase, putative | Pseudomonas syringae pv. |
| | | | phaseolicola 1448A |
| 100 | ZP_02374233.1 | hydrolase, alpha/beta fold family | Burkholderia thailandensis |
| | | protein | TXDOH |
| 101 | YP_003073941.1 | alpha/beta hydrolase family protein | Teredinibacter turnerae |
| | | | T7901 |
| 102 | ZP_00945280.1 | Esterase | Ralstonia solanacearum |
| | | | UW551 |
| 103 | YP_002253305.1 | hydrolase or acyltransferase | Ralstonia solanacearum |
| | | (alpha/beta hydrolase superfamily) | MoIK2 |
| | | protein | |
| 104 | YP_003746098.1 | putative Alpha/beta fold hydrolase | Ralstonia solanacearum |
| | | | CFBP2957 |

INFORMAL SEQUENCE LISTING

SEQ ID NO: 1
Synechococcus elongatus NonA (SYNPCC7002_A1173)
Protein sequence
ST domain is underlined, TE domain is in bold.
MASWSHPQFEKEVHHHHHHGAVGQFANFVDLLQYRAKLQARKTVFSFLADGEAESAALTYGELDQKAQAI
AAFLQANQAQGQRALLLYPPGLEFIGAFLGCLYAGVVAVPAYPPRPNKSFDRLHSIIQDAQAKFALTTTE
LKDKIADRLEALEGTDFHCLATDQVELISGKNWQKPNISGTDLAFLQYTSGSTGDPKGVMVSHHNLIHNS
GLINQGFQDTEASMGVSWLPPYHDMGLIGGILQPIYVGATQILMPPVAFLQRPFRWLKAINDYRVSTSGA
PNFAYDLCASQITPEQIRELDLSCWRLAFSGAEPIRAVTLENFAKTFATAGFQKSAFYPCYGMAETTLIV
SGGNGRAQLPQEIIVSKQGIEANQVRPAQGTETTVTLVGSGEVIGDQIVKIVDPQALTECTVGEIGEVWV
KGESVAQGYWQKPDLTQQQFQGNVGAETGFLRTGDLGFLQGGELYITGRLKDLLIIRGRNHYPQDIELTV
EVAHPALRQGAGAAVSVDVNGEEQLVIVQEVERKYARKLNVAAVAQAIRGAIAAEHQLQPQAICFIKPGS
IPKTSSGKIRRHACKAGFLDGSLAVVGEWQPSHQKEGKGIGTQAVTPSTTTSTNFPLPDQHQQQIEAWLK
DNIAHRLGITPQQLDETEPFASYGLDSVQAVQVTADLEDWLGRKLDPTLAYDYPTIRTLAQFLVQGNQAL
EKIPQVPKIQGKEIAVVGLSCRFPQADNPEAFWELLRNGKDGVRPLKTRWATGEWGGFLEDIDQFEPQFF
GISPREAEQMDPQQRLLLEVTWEALERANIPAESLRHSQTGVFVGISNSDYAQLQVRENNPINPYMGTGN
AHSIAANRLSYFLDLRGVSLSIDTACSSSLVAVHLACQSLINGESELAIAAGVNLILTPDVTQTFTQAGM
MSKTGRCQTFDAEADGYVRGEGCGVVLLKPLAQAERDGDNILAVIHGSAVNQDGRSNGLTAPNGRSQQAV
IRQALAQAGITAADLAYLEAHGTGTPLGDPIEINSLKAVLQTAQREQPCVVGSVKTNIGHLEAAAGIAGL
IKVILSLEHGMIPQHLHFKQLNPRIDLDGLVTIASKDQPWSGGSQKRFAGVSSFGFGGTNAHVIVGDYAQ
QKSPLAPPATQDRPWHLLTLSAKNAQALNALQKSYGDYLAQHPSVDPRDLCLSANTGRSPLKERRFFVFK
QVADLQQTLNQDFLAQPRLSSPAKIAFLFTGQGSQYYGMGQQLYQTSPVFRQVLDECDRLWQTYSPEAPA
LTDLLYGNHNPDLVHETVYTQPLLFAVEYAIAQLWLSWGVTPDFCMGHSVGEYVAACLAGVFSLADGMKL
ITARGKLMHALPSNGSMAAVFADKTVIKPYLSEHLTVGAENGSHLVLSGKTPCLEASIHKLQSQGIKTKP
LKVSHAFHSPLMAPMLAEFREIAEQITFHPPRIPLISNVTGGQIEAEIAQADYWVKHVSQPVKFVQSIQT
LAQAGVNVYLEIGVKPVLLSMGRHCLAEQEAVWLPSLRPHSEPWPEILTSLGKLYEQGLNIDWQTVEAGD
RRRKLILPTYPFQRQRYWFNQGSWQTVETESVNPGPDDLNDWLYQVAWTPLDTLPPAPEPSAKLWLILGD
RHDHQPIEAQFKNAQRVYLGQSNHFPTNAPWEVSADALDNLFTHVGSQNLAGILYLCPPGEDPEDLDEIQ
KQTSGFALQLIQTLYQQKIAVPCWFVTHQSQRVLETDAVTGFAQGGLWGLAQAIALEHPELWGGIIDVDD
SLPNFAQICQQRQVQQLAVRHQKLYGAQLKKQPSLPQKNLQIQPQQTYLVTGGLGAIGRKIAQWLAAAGA
EKVILVSRRAPAADQQTLPTNAVVYPCDLADAAQVAKLFQTYPHIKGIFHAAGTLADGLLQQQTWQKFQT
VAAAKMKGTWHLHRHSQKLDLDFFVLFSSVAGVLGSPGQGNYAAANRGMAAIAQYRQAQGLPALAIHWGP
WAEGGMANSLSNQNLAWLPPPQGLTILEKVLGAQGEMGVFKPDWQNLAKQFPEFAKTHYFAAVIPSAEAV
PPTASIFDKLINLEASQRADYLLDYLRRSVAQILKLEIEQIQSHDSLLDLGMDSLMIMEAIASLKQDLQL
MLYPREIYERPRLDVLTAYLAAEFTKAHDSEAATAAAAIPSQSLSVKTKKQWQKPDHKNPNPIAFILSSP
RSGSTLLRVMLAGHPGLYSPPELHLLPFETMGDRHQELGLSHLGEGLQRALMDLENLTPEASQAKVNQWV
KANTPIADIYAYLQRQAEQRLLIDKSPSYGSDRHILDHSEILFDQAKYIHLVRHPYAVIESFTRLRMDKL
LGAEQQNPYALAESIWRTSNRNILDLGRTVGADRYLQVIYEDLVRDPRKVLTNICDFLGVDFDEALLNPY
SGDRLTDGLHQQSMGVGDPNFLQHKTIDPALADKWRSITLPAALQLDTIQLAETFAYDLPQEPQLTPQTQ
SLPSMVERFVTVRGLETCLCEWGDRHQPLVLLLHGILEQGASWQLIAPQLAAQGYWVVAPDLRGHGKSAH
AQSYSMLDFLADVDALAKQLGDRPFTLVGHSMGSIIGAMYAGIRQTQVEKLILVETIVPNDIDDAETGNH
LTTHLDYLAAPPQHPIFPSLEVAARRLRQATPQLPKDLSAFLTQRSTKSVEKGVQWRWDAFLRTRAGIEF
NGISRRRYLALLKDIQAPITLIYGDQSEFNRPADLQAIQAALPQAQRLTVAGGHNLHFENPQAIAQIVYQ
QLQTPVPKTQGLHHHHHHSAWSHPQFEK
SEQ ID NO:2
Synechococcus elongatus NonA (SYNPCC7002_A1173)
ST domain protein sequence
FILSSPRSGSTLLRVMLAGHPGLYSPPELHLLPFETMGDRHQELGLSHLGEGLQRALMDLENLTPEASQA
KVNQWVKANTPIADIYAYLQRQAEQRLLIDKSPSYGSDRHILDHSEILFDQAKYIHLVRHPYAVIESFTR
LRMDKLLGAEQQNPYALAESIWRTSNRNILDLGRTVGADRYLQVIYEDLVRDPRKVLTNICDFLGVDFDE
ALLNPY
SEQ ID NO:3
Synechococcus elongatus NonA (SYNPCC7002_A1173)
TE domain protein sequence
FVTVRGLETCLCEWGDRHQPLVLLLHGILEQGASWQLIAPQLAAQGYWVVAPDLRGHGKSAHAQSYSMLD
FLADVDALAKQLGDRPFTLVGHSMGSIIGAMYAGIRQTQVEKLILVETIVPNDIDDAETGNHLTTHLDYL
AAPPQHPIFPSLEVAARRLRQATPQLPKDLSAFLTQRSTKSVEKGVQWRWDAFLRTRAGIEFNGISRRRY
LALLKDIQAPITLIYGDQSEFNRPADLQAIQAALPQAQRLTVAGGHNLHFENPQAIAQIV

1. An engineered microbial cell for producing a hydrocarbon, wherein said engineered microbial cell comprises a recombinantly expressed protein selected from Tables 1-3 (SEQ ID NOS 4-104, respectively, in order of appearance), and wherein said cell synthesizes at least one terminal olefin.

2.-7. (canceled)

8. The engineered microbial cell of claim 1, wherein said at least one terminal olefin is propylene.

9. The engineered microbial cell of claim 1, wherein said engineered microbial cell comprises 3-hydroxybutyryl-ACP.

10. (canceled)

11. The engineered microbial cell of claim 9, wherein said engineered microbial cell comprises a recombinant accBCAD gene or a recombinant fabDHG gene.

12. The engineered microbial cell of claim 9, wherein said engineered microbial cell comprises a recombinant 3-hydroxyacyl ACP dehydratase gene, wherein said gene comprises a modification that reduces its expression, comprises a knock-out mutation, or is under the control of an inducible promoter.

13.-14. (canceled)

15. The engineered microbial cell of claim 1, wherein said engineered microbial cell comprises hydroxybutyryl-CoA.

16. (canceled)

17. The engineered microbial cell of claim 7, wherein said engineered microbial cell comprises a recombinant phaA gene or a recombinant phaB gene.

18. (canceled)

19. The engineered microbial cell of claim 3, wherein said propylene is synthesized from acetyl-CoA.

20. The engineered microbial cell of claim 1, wherein said at least one terminal olefin is selected from the group consisting of: ethylene, propylene, butylene, butadiene, isoprene, and 1-nonadecene.

21. The engineered microbial cell of claim 1, wherein said engineered microbial cell comprises a recombinant curM gene.

22. The engineered microbial cell of claim 1, wherein said engineered microbial cell comprises a recombinant nonA gene.

23.-51. (canceled)

52. The engineered microbial cell of claim 1, wherein said recombinantly expressed protein comprises a recombinant sulfotransferase protein activity and/or a recombinant thioesterase protein activity.

53. A method for producing a terminal olefin, comprising:
a. culturing an engineered microbial cell in a culture medium, wherein said engineered microbial cell comprises a recombinantly expressed protein selected from Tables 1-3 (SEQ ID NOs: 4-104, respectively, in order of appearance), and wherein said cell synthesizes at least one terminal olefin.
b. isolating said terminal olefin from said microbial cell or said culture medium.
